(19) United States
(12) Patent Application Publication
(10) Pub. No.: US 2024/0116843 A1
(43) Pub. Date: Apr. 11, 2024

(54) PROCESS FOR THE PREPARATION OF VITAMIN K2 AND NOVEL INTERMEDIATES

(71) Applicant: KAPPA BIOSCIENCE AS, Oslo (NO)

(72) Inventors: Marcel SANDBERG, Oslo (NO); Inger Reidun AUKRUST, Oslo (NO)

(21) Appl. No.: 18/280,356

(22) PCT Filed: Mar. 7, 2022
(86) PCT No.: PCT/EP2022/055742

§ 371 Date: Sep. 5, 2023

(30) Foreign Application Priority Data
Mar. 5, 2021 (GB) 2103137.2

(51) Int. Cl. C07C 46/00; C07C 50/22

(52) U.S. Cl. CPC C07C 46/00; C07C 50/22; C07C 2602/10; C07C 2603/86

(57) ABSTRACT

A compound of formula (I): (I) wherein n is an integer between 0 and 10; Y is hydrogen, halide, mesylate, tosylate, ester, —SPh, —SO2Ph, hydroxyl or protected hydroxyl; with the proviso that if n=0 or 1, Y is not hydrogen.

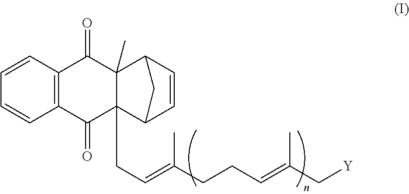

This application relates to an intermediate in the synthesis of vitamin K2 compounds, as well as to a process for the synthesis of vitamin K2 compounds.

Vitamin K denotes a group of lipophilic and hydrophobic vitamins that are needed for the post-translational modification of certain proteins, mostly required for blood coagulation. Chemically they are 2-methyl-1,4-naphthoquinone derivatives.

Vitamin K is not a single compound, rather it is a series of related analogues. Vitamin K1 is called phylloquinone and has the systematic name all-E-2-methyl-3-(3,7,11,15-tetramethylhexadec-2-enyl) naphthalene-1,4-dione. Vitamin K2 is a menaquinone.

Vitamin K2 is a mixture of different molecules based on a naphthoquinone structure and varying lengths of isoprenoid chains. The compound MK-7 (i.e. 7 isoprenyl groups) is depicted below but other components of the vitamin have different numbers of isoprenoid links. Menaquinones have side chains composed of all-E polyprenyl residues; generally, they are designated as MK-n, where n specifies the number of isoprenoid repeating units. The minimum value of n is 2.

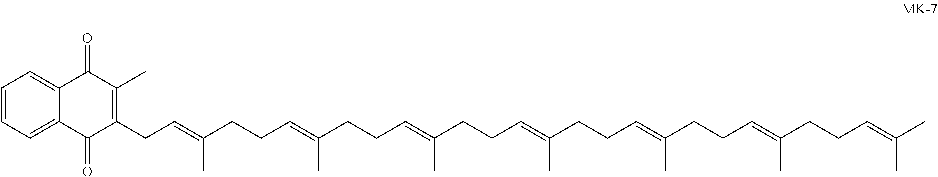

It is known that γ-carboxylated osteocalcin is involved in the bone remodelling system, and there are strong indications that vitamin K has a beneficial effect on bone diseases such as osteoporosis. There is interest therefore in the investigation of various vitamin K2 type compounds for inhibitory effects on osteoporosis. Vitamin K2 has also been implicated in various medical applications including as potential anti-cancer drugs and for beneficial cardio-vascular activity.

Whilst vitamin K2 occurs naturally in low concentrations in various fermented food products such as cheese and can to a small extent be produced by bacteria in the intestines, its use as a dietary supplement may be beneficial for many populations. Vitamin K2 can be produced by fermentation of soy beans, but it is still an interesting synthetic target as isolation of the vitamin from a natural source is complex and concentrations of the vitamin are low. Moreover, synthesis allows the preparation of particular menaquinones rather than the isolation of a mixture of different menaquinones.

Various individuals have synthesized the menaquinone compounds which form part of vitamin K2 or components thereof. The first synthesis of menaquinones, reported by Isler et al., Helv. Chim Acta 1958, 41, 786-807, used a nonstereospecific approach. Tso and Chen, J Chem Res 1995, 104-105 describes a one-pot synthesis of vitamin K although he concentrates on the formation of the naphthoquinone ring as opposed to the side chain of the molecule. His chemistry involves the reaction of 3-substituted isobenzofuranones with vinylic sulphones to form the naphthoquinone ring structure.

Suhara et al, Bioorg Med Chem Lett 17, (2007) 1622-1625, describe various syntheses of menaquinone analogues in which the terminal methyl group is converted to a hydroxyl, aldehyde or acid group.

Naruta, J Org Chem 1980, 45, 4097-4104, describes the synthesis of some vitamin K2 analogues using trialkylallylstannane chemistry to bond the preformed side-chain to the naphthoquinone group.

A paper by Suhara et al. (Biorganic Med. Chem Lett. 17 (2007) 1622-1625) suggests the use of certain vitamin K metabolites as biologically active compound which can lead to the development of new drugs based on side-chain modification of the alkyl group. Suhara targets compounds of structure similar to metabolites of vitamin K, i.e. compounds carrying acidic (or other strongly hydrophilic groups) at the terminus of the 3-position side chain. Some of the analogues have apoptosis-inducing properties.

The present inventors have previously devised a synthetic strategy for the formation of MK-7 and other menaquinones involving the synthesis of a key intermediate in the manufacturing process (WO2010/035000). This process enables the formation of large synthetic quantities of vitamin K2 not previously enabled in the prior art. This synthesis relies on Kumada and Suzuki coupling which require —B(OH)₂ and —MgHal groups on the menadione respectively, as well as transition metal catalysts. The polyisoprene chain is also constructed stepwise using Bielmann couplings, which also requires a separate reduction of —SPh and —SO₂Ph groups in the resulting polyisoprene chain.

Whilst this process is effective, it uses a number of steps and on industrial scale an improved process is desirable.

The present inventors sought an alternative process for making vitamin K2. The present inventors now propose a process involving a retro Diels-Alder reaction. Teitelbaum et al. (Synthesis 2015, 47, 944-948) disclosed the synthesis of certain vitamin K chain-shortened acid metabolites using a menadione Diels-Alder adduct. The synthesis requires the retro-Diels-Alder reaction from a polyisoprene-substituted menaquinone Diels-Alder adduct. The retro-Diels-Alder step is performed in the presence of an acid and ammonium catalyst. Moreover, the Teitelbaum process prepares an acid metabolite via an epoxy and then aldehyde intermediate.

Ji (Synthetic Communications 2003, 33:5, 763-772) teaches Diels-Alder chemistry similar to Teitelbaum's, but the focus is on vitamin K1 compounds. The retro-Diels-Alder reaction is performed on a phytyl-substituted menadione Diels Alder adduct. Vitamin K1 does not have the polyunsaturation in the long phytyl chain.

The present inventors have devised an improved synthetic strategy for the formation of MK-7 and other long chain menaquinones. The method relies on a retro Diels-Alder reaction from a particular intermediate formed from a menaquinone Diels-Alder adduct and a polyisoprene source. The intermediate itself is of particular interest as it is a stable compound which can undergo near-quantitative conversion to MK-7 or other long-chain menaquinones upon simple heating or upon reaction with an ammonium and/or acid catalyst. The reaction can be done both in batch and in flow and does not require any intermediate purification steps. The target menaquinones can be prepared in high yield and with stereochemical integrity.

The retro-Diels-Alder reaction described in Teitelbaum and Ji (see above) using ammonium and acid catalysts can be employed for longer chain compounds as well however this is less favourable. Also, the use of dodecyltrimethylammonium bromide and acetic acid lowers the yield of product and increases the amount of undesired cis-isomers (from around 6 to around 12%). Heating to effect the retro Diels-Alder reaction is advantageous. The use of these reagents is more expensive, and we observe that the yield is lower and the risk of bond racemisation higher. Furthermore, the Diels-Alder route to menaquinones can also be adapted such that the polyisoprene chain extension chemistry described in WO2010/035000 can be employed.

### SUMMARY OF THE INVENTION

Viewed from a first aspect the invention provides a compound of formula (I):

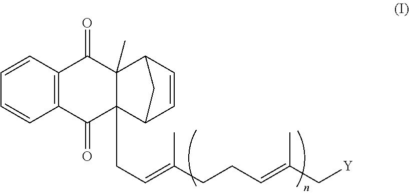

wherein n is an integer between 0 and 10;
Y is hydrogen, halide, mesylate, tosylate, —SPh, —SO₂Ph, hydroxyl or protected hydroxyl;
with the proviso that if n=0 or 1, Y is not hydrogen.

Viewed from another aspect the invention provides a compound of formula (I′):

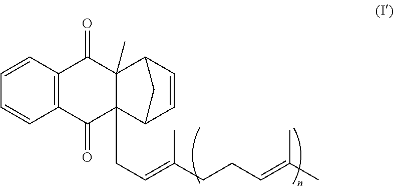

wherein n is an integer between 2 and 10;

Viewed from another aspect the invention provides a process in which a compound of formula (I′)

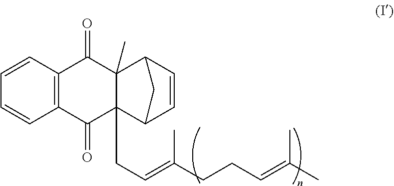

wherein n is an integer between 2 and 10; is converted into a compound of formula (VII):

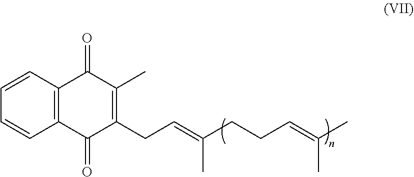

preferably by heating, especially in the absence of a solvent.

Viewed from another aspect, the invention provides a process which comprises converting a compound of formula (I″)

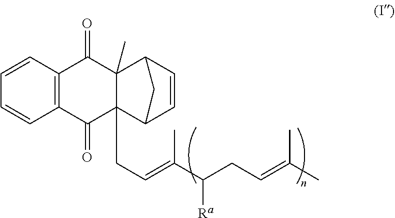

wherein n is an integer between 2 and 10
and wherein R^{a} is independently H or a substituent selected from mesylate, tosylate, —SPh or —SO₂Ph
   to a compound of formula (VII″)

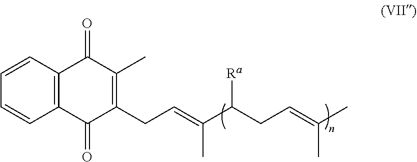

preferably by heating, especially in the absence of a solvent.

Viewed from another aspect, the invention provides a compound of formula (I″)

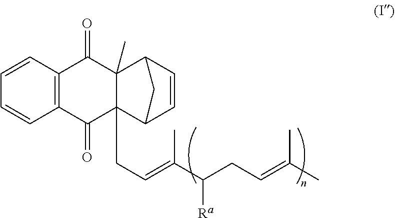

wherein n is an integer between 2 and 10
and wherein R^{a} is independently H or a substituent selected from mesylate, tosylate, —SPh or —SO₂Ph.

Viewed from another aspect, the invention provides a compound of formula (I′″):

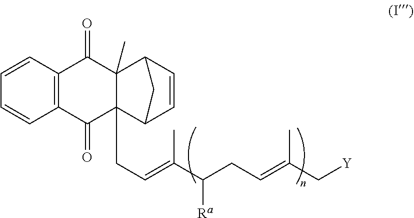

wherein R^{a} is independently H or a substituent selected from mesylate, tosylate, —SPh or —SO₂Ph;
Y is hydrogen, halide, mesylate, tosylate, —SPh, —SO₂Ph, ester, hydroxyl or protected hydroxyl
n is an integer between 0 and 10 when R^{a} is H, and n is an integer between 2 and 10 when Y is H;
with the proviso that if n=0 or 1, Y is not hydrogen.

The features of the aspects and/or embodiments indicated herein are usable individually and in combination in all aspects and embodiments of the invention where technically viable, unless otherwise indicated.

### Definitions

A polyprenyl side chain is one which derives from the polymerisation of isoprene: 2-methyl-1,3-butadiene. A ‘polyprene’ or polyisoprene’ chain contains at least 2 polymerised units of isoprene.

Unless otherwise stated, an alkyl group can contain 1 to 6 carbon atoms, preferably 1 to 4 carbon atoms, especially methyl or ethyl. In all embodiments R is preferably methyl.

Halide (Hal) includes fluoro, chloro, bromo and iodo, preferably chloro or bromo, most preferably bromo.

The term leaving group is well known in the art and denotes an atom or group of atoms that readily leaves a molecule due to the relative stability of the ion formed. Useful leaving groups include halides, tosylates, mesylates and triflates.

In any embodiment of the invention, preferred leaving groups are halides, in particular bromine.

Mesylate is OSO₂Me.

Tosylate is OSO₂Ph-Me.

### DETAILED DESCRIPTION OF THE INVENTION

This invention provides a new synthetic route to vitamin K2 and to the menaquinones that form part of naturally occurring vitamin K2. The invention also relates to new intermediate compounds that are prepared during the synthesis. The Diels-Alder adducts of the invention are preferably precursors to MK-4, MK-6, MK-7, MK-8, or MK-10. Most preferably, they are precursors to MK-7 and n is 6. It is thus preferred if the long chain isoprenoid at position 2 on the naphthoquinone ring is

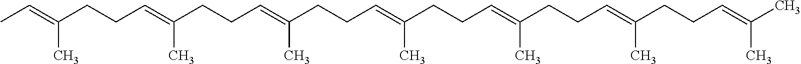 [SMILES: C/C=C(\C)CC/C=C(\C)CC/C=C(\C)CC/C=C(\C)CC/C=C(\C)CC/C=C(\C)CCC=C(C)C]

### Diels-Alder Intermediate and its Formation

In a first aspect of the invention, the invention provides a Diels-Alder (DA) adduct of formula (I):

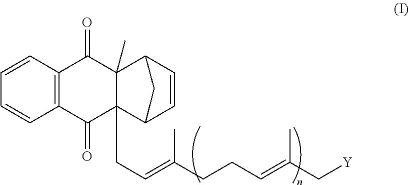

wherein n is an integer between 0 and 10,
Y is hydrogen, halide, mesylate, tosylate, —SPh, —SO₂Ph, ester, hydroxyl or protected hydroxyl;
with the proviso that if n=0 or 1, Y is not hydrogen.

The ester is typically of formula —COOR¹ with R¹ being a C₁-C₆ hydrocarbyl group, such as a linear or branched C₁-C₆ alkyl group or Ph. Typically R¹ is methyl and the ester group is acetate.

By protected hydroxyl is meant an OH group protected by a protecting group. Protected hydroxyl is thus typically an —OR group wherein R is MOM, THP, tBu, allyl, benzyl, TBDMS, TBDPS, —C(═O)tBu, —C(═O)Ph.

The integer n is typically between 2 and 10, preferably between 3 and 9. Preferably the compound of formula (I) is a Diels-Alder Adduct of MK-4, MK-6, MK-7, MK-8 or MK-10, i.e. n is 3, 5, 6, 7, 9. Most preferably n is 6 (i.e. the Diels-Alder adduct (1) is a precursor to MK-7). When Y is hydrogen, n is typically in the range 5-10, 5-9, 5-8, or 5-7, preferably 5-7, most preferably 6. The above definitions for n also apply at least to compounds of formula (I′), (I″) and (I′″), where technically viable, and unless stated otherwise.

Longer chains, i.e. higher values of n, are surprisingly stable, e.g. during the retro Diels Alder step, and enable the facile preparation of higher MK-n compounds.

In one embodiment, e.g. where n is 0 or 1, the functionality at Y can be used to attach a further polyprenyl, such as a pentaprenyl substituent in a ‘[2+5]’ reaction or a hexaprenyl substituent in a ‘[1+6]’ reaction. More preferably Y is H and the compound of formula (I) becomes:

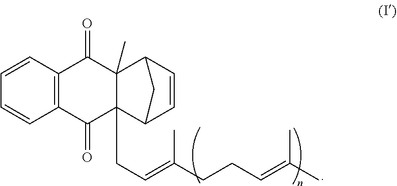

wherein n is 2 to 10, such as 3 to 9, or 5-10, 5-9, 5-8, or 5-7, especially 6.

Teitelbaum et al. (Synthesis 2015, 47, 944-948) describes the above compound (1) in which n is 0 or 1 and Y═H, but these intermediates are then further reacted to form short chain acid metabolites.

The presence of a Y functionality (i.e. when Y is not hydrogen) enables chain lengthening chemistry to be performed at the end of the polyisoprene chain once the attachment to menaquinone Diels-Alder adduct has been performed. Thus, the Diels-Alder intermediate (1) can be converted directly to the final menaquinone or can undergo further chain lengthening at Y before the retro DA reaction.

In a further embodiment, a compound of formula (I″) is provided:

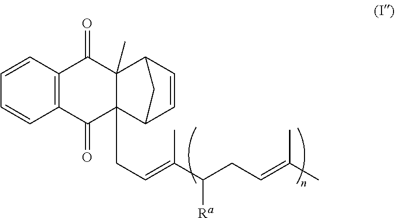

wherein n is an integer between 2 and 10, such as 3 to 9, especially 6. and wherein Ra is independently H or a substituent selected from mesylate, tosylate, —SPh or —SO₂Ph.

The Ra group(s) are typically present when the polyisoprene chain has been constructed using Bielmann chemistry. The retro Diels Alder reaction can be performed after removing the R^{a} group(s), or before.

The compounds of formula (I) and (I″) can be combined to give the following compound definition (I′″), which forms a further embodiment of the invention:

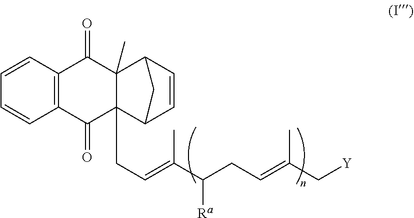

wherein R^{a} is independently H or a substituent selected from mesylate, tosylate, —SPh or —SO₂Ph;
Y is hydrogen, halide, mesylate, tosylate, —SPh, —SO₂Ph, ester, hydroxyl or protected hydroxyl
n is an integer between 0 and 10 when R^{a} is H, and n is an integer between 2 and 10 when Y is H;
with the proviso that if n=0 or 1, Y is not hydrogen.

The Diels-Alder adduct of formula (I) of the invention is typically prepared from the reaction of a compound of formula (II) and (Ill)

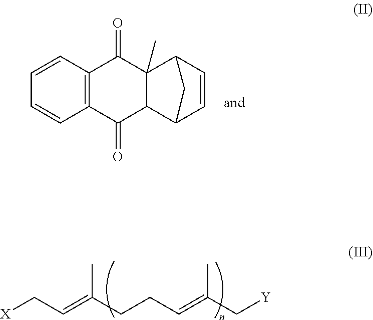

in the presence of a base;
wherein X is a leaving group preferably selected from halogen, mesylate, tosylate and triflate. X is preferably a halogen, and most preferably Br. Y can be as defined for compound (1). Preferably, Y is hydrogen, mesylate, tosylate, —SPh, —SO₂Ph, ester, hydroxyl or protected hydroxyl. In a particular embodiment, Y is an ester, such as an ester of formula —COOR¹, with R¹ being a C1-C6 hydrocarbyl group, such as a linear or branched C1-C6 alkyl group or Ph.

Any base can be used, typically a non-nucleophilic base. The base is typically an alkoxide, such as a tert-butoxide (e.g. KO^{t}Bu). The base deprotonates the hydrogen in compound (II) which is alpha to the bottom carbonyl group. The base is typically added once compound (II) and compound (Ill) have been mixed; however, it may also be added to compound (II) before addition of compound (Ill).

Typically, Y is H in all embodiments of the invention. Thus, in a particular embodiment of the invention, no chain extension chemistry as described herein or below is performed. In such an embodiment, the invention provides a compound of formula:

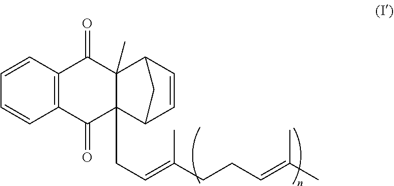

wherein n is an integer between 2 and 10, preferably between 2 and 8. Preferably compound (I′) is a Diels-Alder Adduct of MK-4, MK-6, MK-7, MK-8 or MK-10, i.e. n is 3, 5, 6, 7, 9. Preferably n is 6 (i.e. the Diels-Alder intermediate (IV) is a precursor to MK-7). These preferences are valid for all embodiments of the invention, where technically viable. I.e. these preferred values of n also apply to any embodiments relating to other compounds or processes described herein.

The integer n is preferably 6, and thus in a preferred embodiment the invention provides a compound of formula:

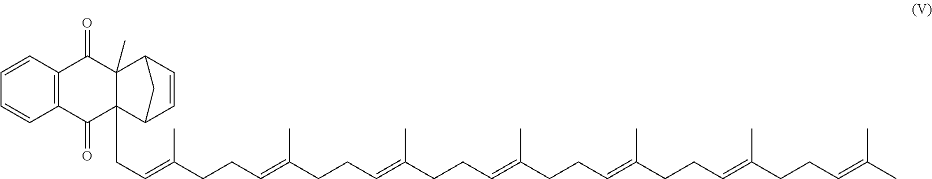 [SMILES: CC(C)=CCC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1]

Typically, the compound of formula (I′) is formed from the reaction of a compound of formula (II) and (III′)

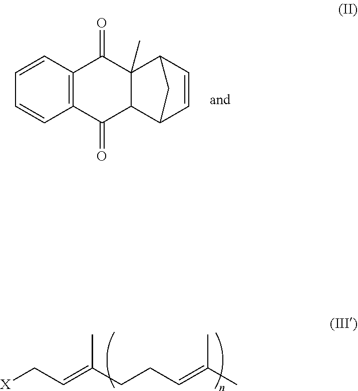

in the presence of a base;
wherein X is a leaving group preferably selected from halogen, mesylate, tosylate and triflate and n is an integer between 2 and 10, preferably between 2 and 8 . . . X may also be an ester, such as an ester of formula —COOR¹, with R¹ being a C1-C6 hydrocarbyl group, such as a linear or branched C1-C6 alkyl group or Ph. It is envisaged that such polyprene esters may react with the menadione Diels-Alder adduct (II) in the presence of a base. X is preferably a halogen, and most preferably Br.

Diels-Alder adduct (V) is preferably formed from the menaquinone adduct (II)

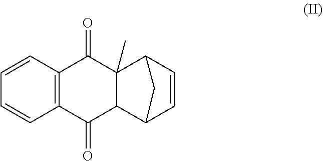 [SMILES: CC12C(=O)c3ccccc3C(=O)C1C1C=CC2C1]

and the polyprene reagent:

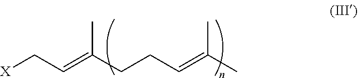

wherein n is 6;
in the presence of a base;
wherein X is a leaving group selected from halogen, mesylate, tosylate and triflate. X may also be an ester, such as an ester of formula —COOR¹, with R¹ being a C1-C6 hydrocarbyl group, such as a linear or branched C1-C6 alkyl group or Ph. It is envisaged that such polyprene esters may react with the menadione adduct (II) in the presence of a base. X is preferably a halogen, and most preferably Br.

Other preferred adducts of formula (I′) include:

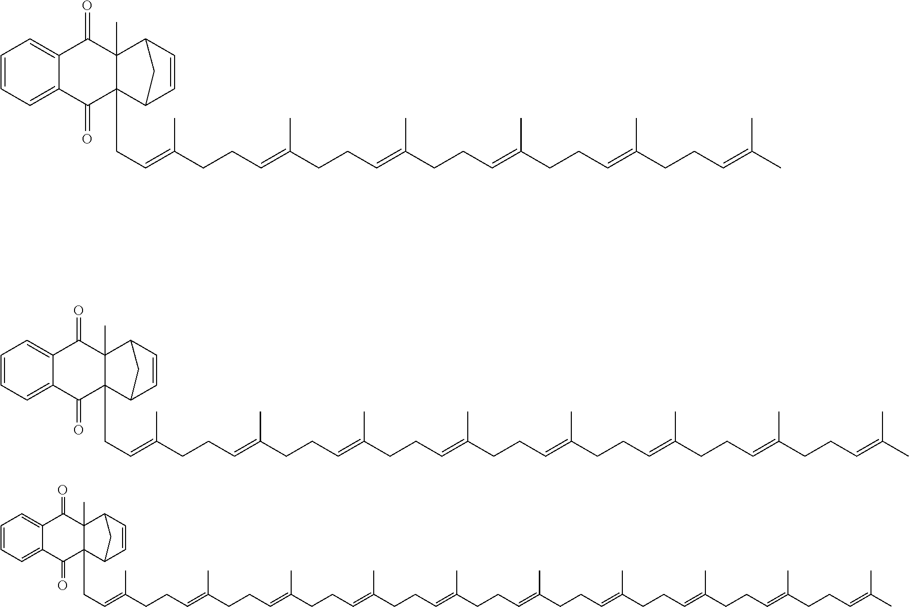 [SMILES: CC(C)=CCC/C(C)=C/CC/C(C)=C/CC/C(C)=C/CC/C(C)=C/CC/C(C)=C/CC12C(=O)c3ccccc3C(=O)C1(C)C1C=CC2C1 ; CC(C)=CCC/C(C)=C/CC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1 ; CC(C)=CCC/C(C)=C/CC/C(C)=C/CC/C(C)=C/CC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1]

### Chain Lengthening Using Bielmann Chemistry

As mentioned above, the chain can be further lengthened, typically using Bielmann chemistry. In a particular embodiment, the invention provides a process whereby a compound of formula (IV):

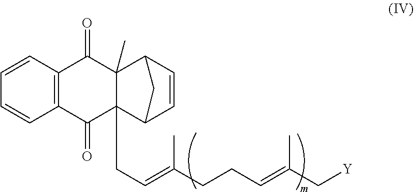

is (i) reacted with a compound of formula (VI):

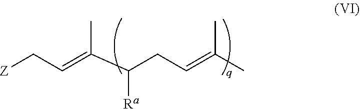

in the presence of a base,
wherein m+q=n−1;
n is 1 to 9;
m is an integer between 0 and 9;
q is an integer between 9 and 0;
Y is halide, hydroxyl or protected hydroxyl and Z is mesylate, tosylate, ester, —SPh or —SO₂Ph, or Y is mesylate, tosylate, ester, —SPh or —SO₂Ph and Z is halide, hydroxyl or protected hydroxyl;
R^{a} is independently H or a substituent selected from mesylate, tosylate, —SPh or —SO₂Ph;
   and optionally in a step ii), the Z or Y group respectively and any non-H R^{a} group in the resulting compound are reduced to hydride(s). In a further embodiment, the Z or Y group respectively and any non-H R^{a} group in the resulting compound are reduced to hydride(s) after the retro-Diels Alder reaction.

In a particular embodiment, m is 1 and q is 4. In a further embodiment, m is 0 and q is 5. Both of these lead to MK-7-based structures. Typically, m is 0-4 and q is 6-2, preferably m is 0-2 and q is 5-3.

Preferably, n is 6. Typically, therefore, m+q=5.

In a further embodiment, Y is —SO₂Ph or —SPh and Z is a halide (e.g. Br).

In a particular embodiment, the reduction in step ii) is performed using lithium metal or a metal hydride.

The compound resulting from step (i) is typically the following intermediate (Iᵢₙₜ):

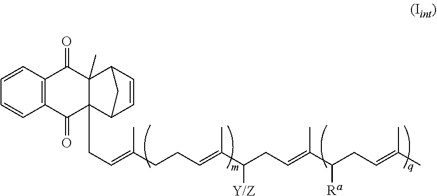

If Y is halide, hydroxyl or protected hydroxyl and Z is mesylate, tosylate, —SPh or —SO₂Ph, then ‘Y/Z’ will be Z. If Y is mesylate, tosylate, —SPh or —SO₂Ph and Z is halide, hydroxyl or protected hydroxyl, then ‘Y/Z’ will be Y. Typically, in compound (IV), Y is mesylate, tosylate, —SPh or —SO₂Ph and in compound (VI), Z is halide, hydroxyl or protected hydroxyl, most preferably Y is —SPh or —SO₂Ph (preferably —SO₂Ph) and Z is halide (preferably Br).

In compound (VI) (and (Iᵢₙₜ)), the R^{a} group can be different for each repeating unit. In a particular embodiment, at least one R^{a} substituent, preferably one R^{a} substituent, is selected from mesylate, tosylate, —SPh or —SO₂Ph, and the other R^{a} groups are H. In another embodiment, all R^{a} groups are H. Any non-H R^{a} groups in compound (VI) typically come from the construction chemistry used to form compound (VI) (typically Bielmann chemistry).

Typically, step (ii) produces compound (I′). In a particular embodiment, therefore, Iᵢₙₜ is reduced in step (ii) to form (I′):

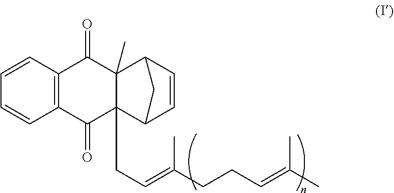

Alternatively, the retro Diels-Alder reaction is performed on Iᵢₙₜ to form

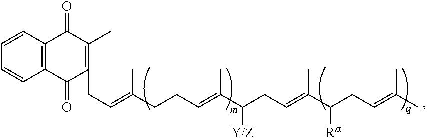

after which step ii) is performed (i.e. the Z or Y group respectively and any non-H R^{a} group in the resulting compound are reduced to hydride(s)).

Alternatively viewed, the invention provides the following process which comprises converting a compound of formula (I″)

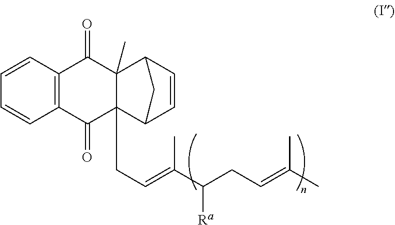

wherein n is an integer between 2 and 10
and wherein R^{a} is independently H or a substituent selected from mesylate, tosylate, —SPh or —SO₂Ph
   to a compound of formula (VII″):

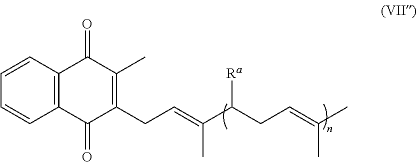

preferably by heating.

The compound (I″) per se forms an embodiment of the invention.

To perform the chain extension coupling of step i), Biellmann chemistry can preferably be employed. This reaction involves the formation of phenylthio or phenylsulfonyl substituted compounds and reaction of these sulphur compounds with an electrophile, such as a halide, in the presence of a base. Suitable bases include n-butyl lithium, tert butyl lithium, and non nucleophilic bases such as tertbutoxides. After coupling, the phenylthio or phenylsulfonyl groups are removed reductively for example with lithium metal or a metal hydride. Alternatively, the phenylthio or phenylsulfonyl groups are removed reductively, for example with lithium metal or a metal hydride, after the retro-Diels-Alder reaction. The inventors have found that this method is ideal for coupling together two isoprenoid chains to make a new isoprenoid chain of, for example, 7 or more units.

The chemistry here is previously explained in WO2010/03500. Thus in a particular embodiment, the invention provides a process represented by the following scheme:

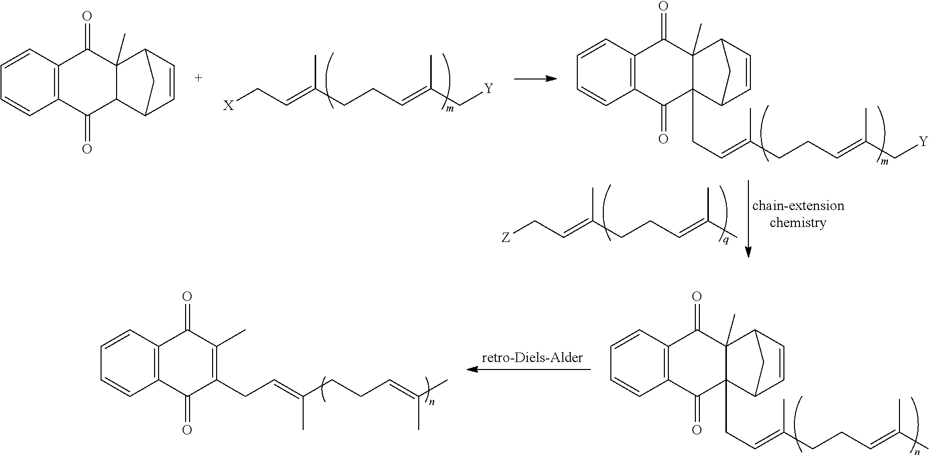

In a particular embodiment, the invention provides the following process (i.e. a ‘[1+6]’ process):

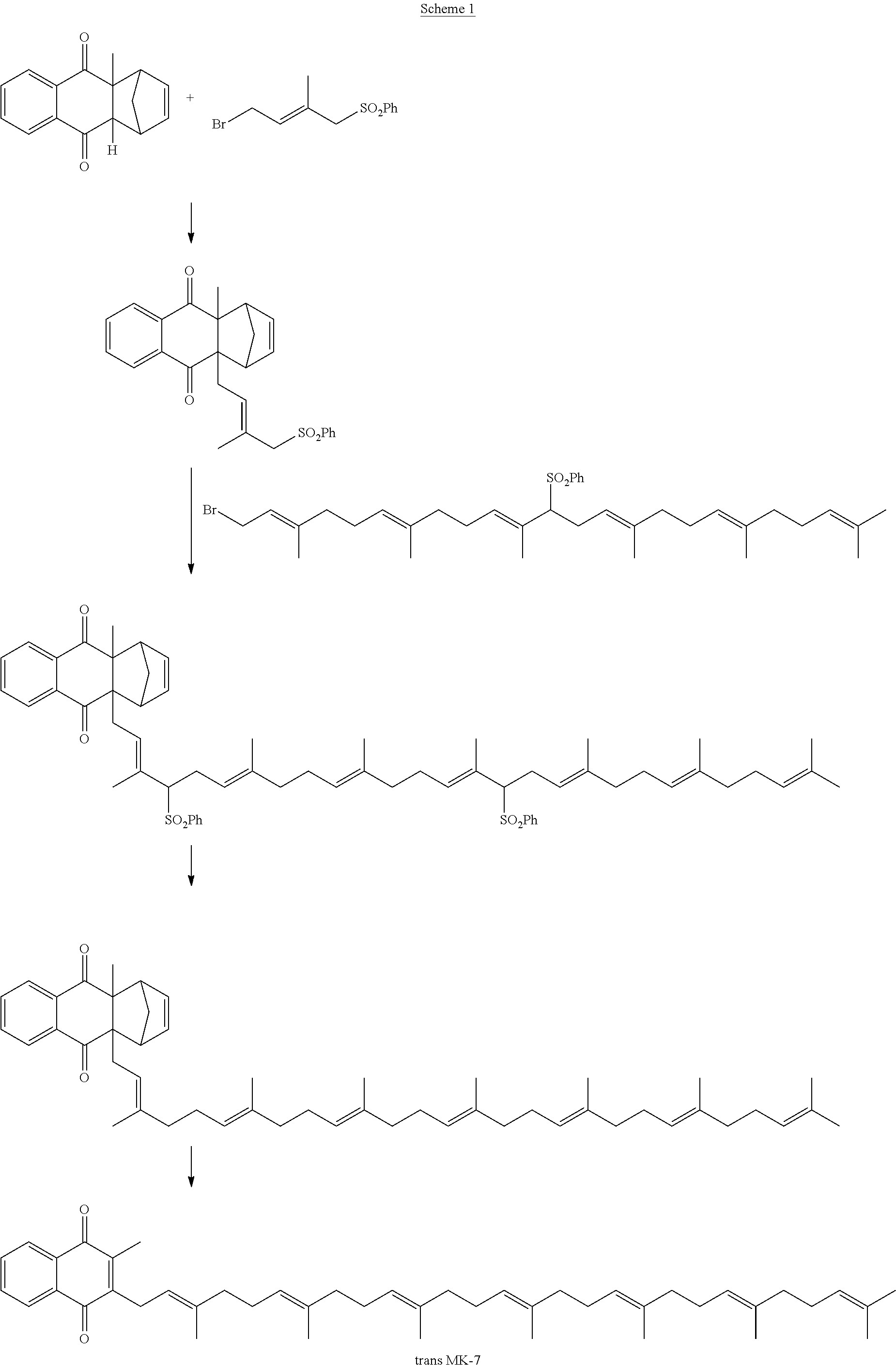

or

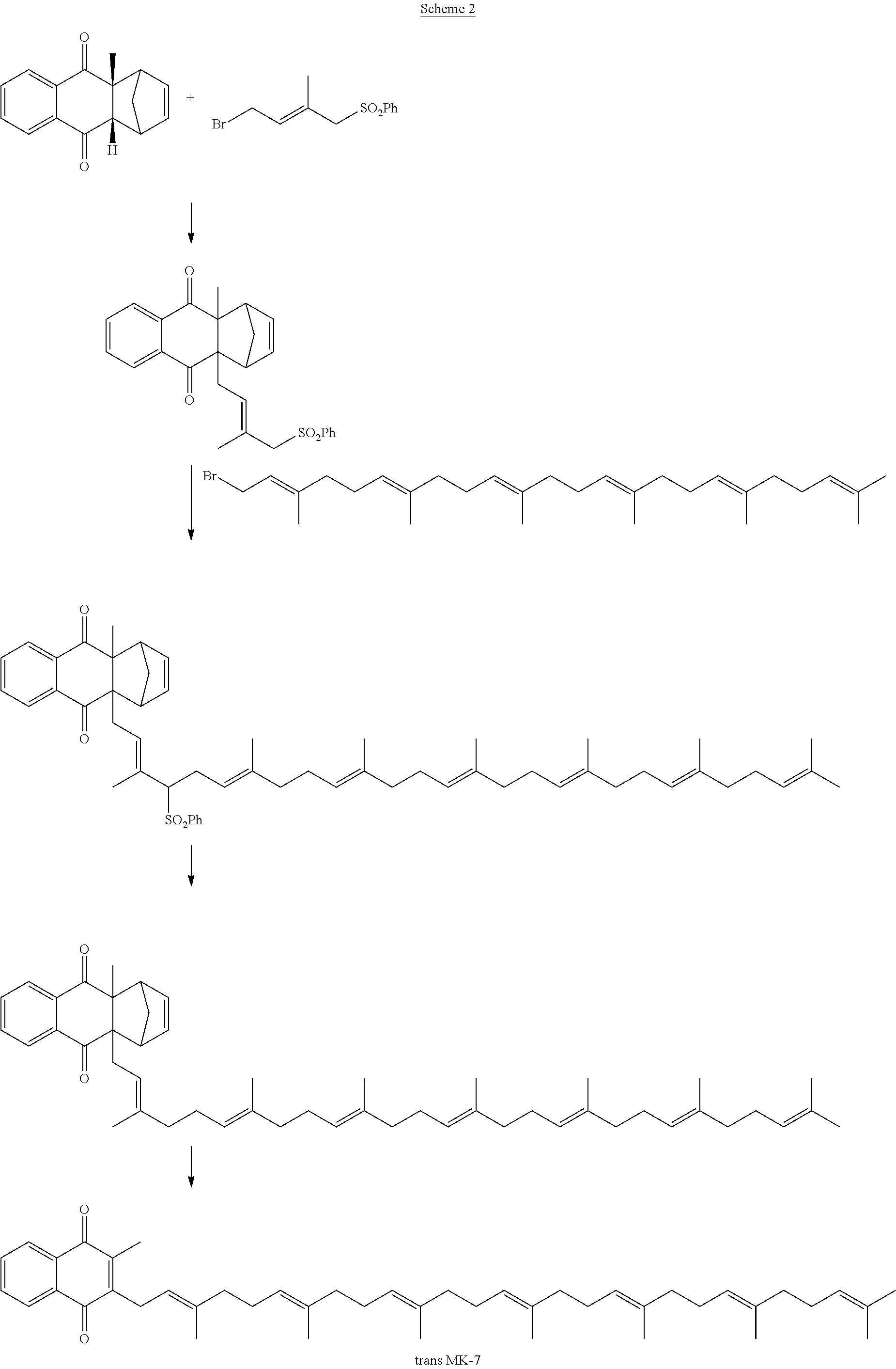

In a further particular embodiment, the invention provides the following process (i.e. a ‘[2+5]’ process):

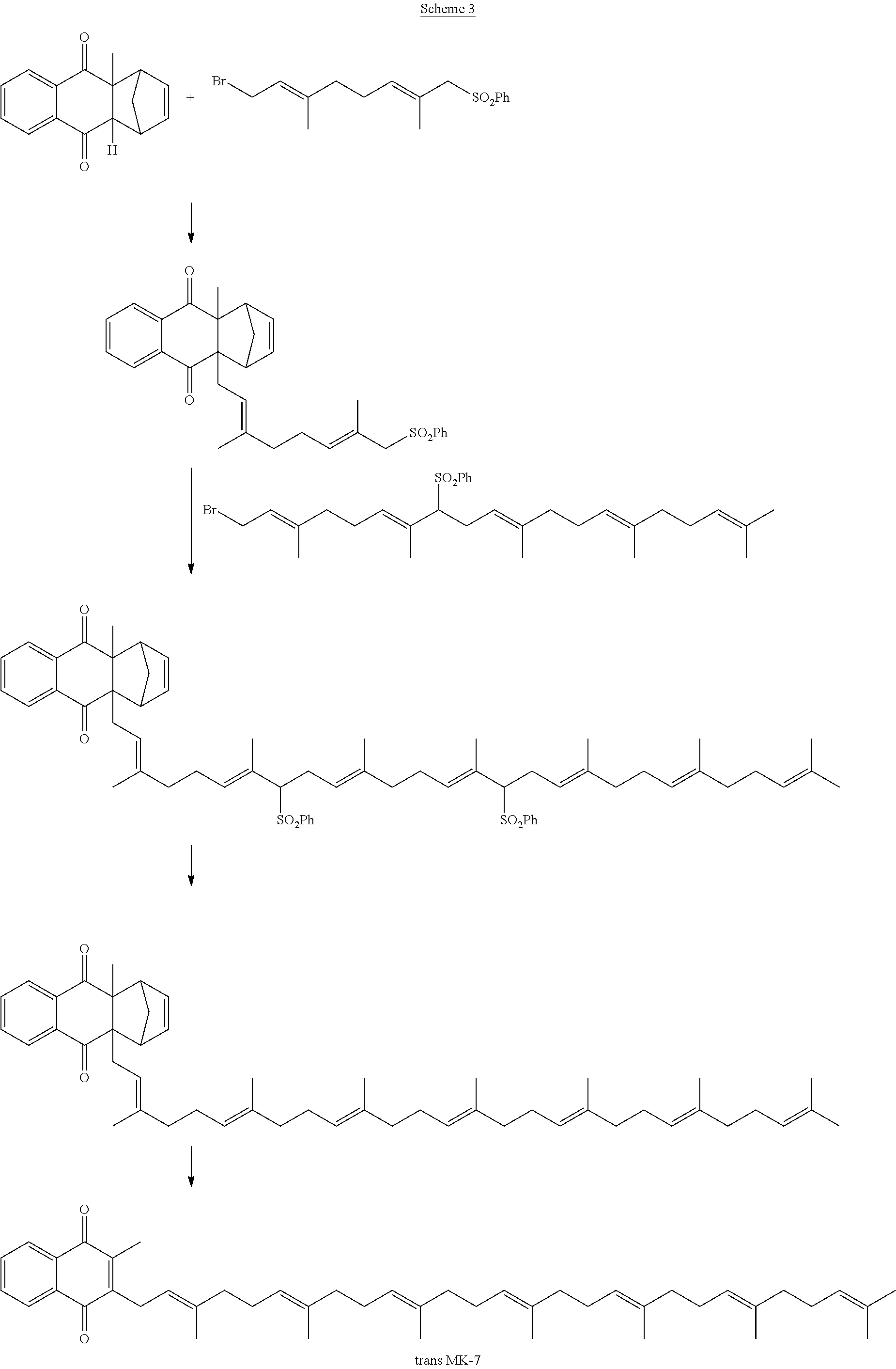

or

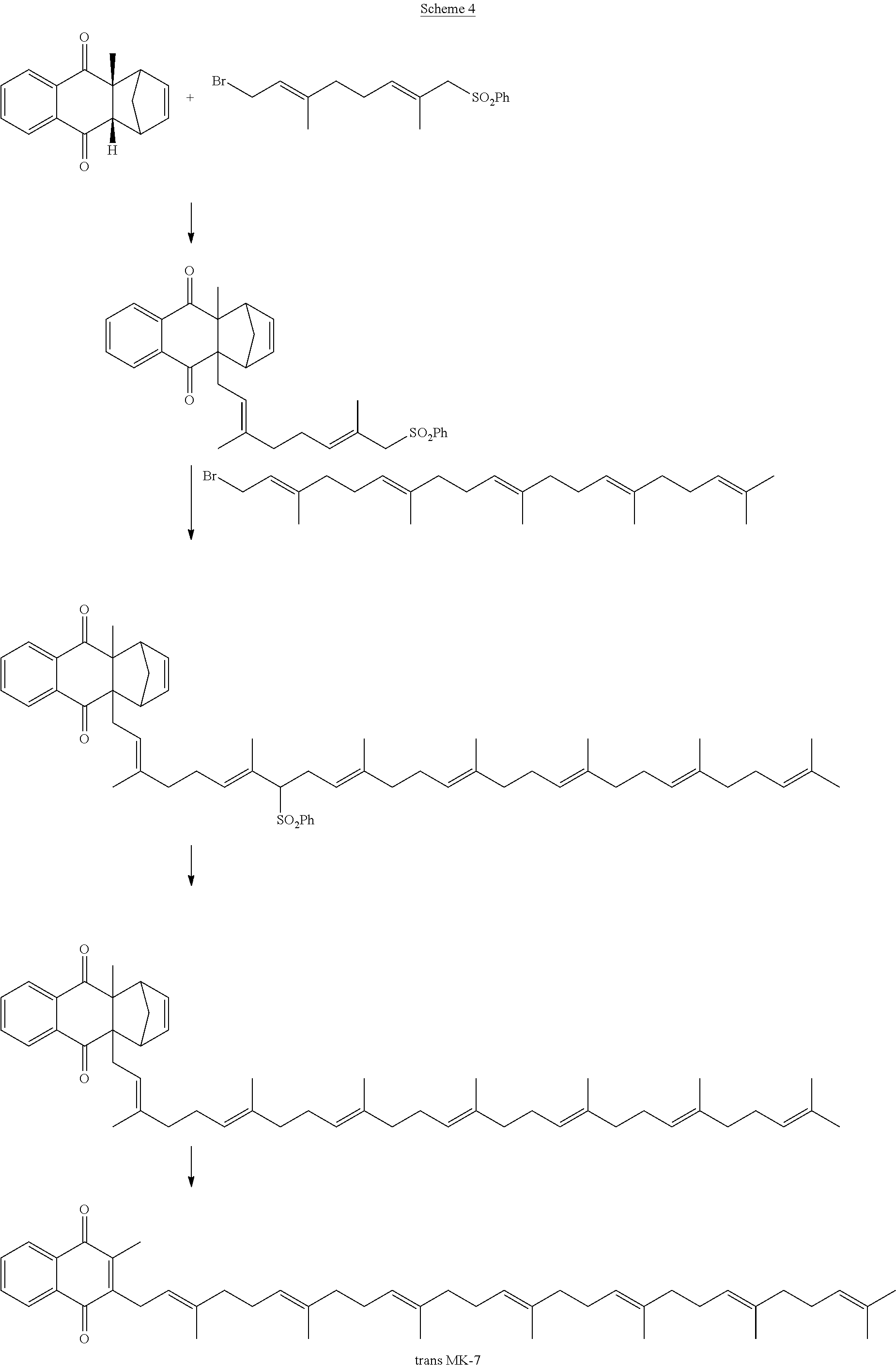

In any of the above schemes 1-4, the last two steps can be interchanged, i.e. the retro Diels-Alder can be performed before the removal of the —SO₂PH groups. For scheme 1, for example, the last two steps would then be:

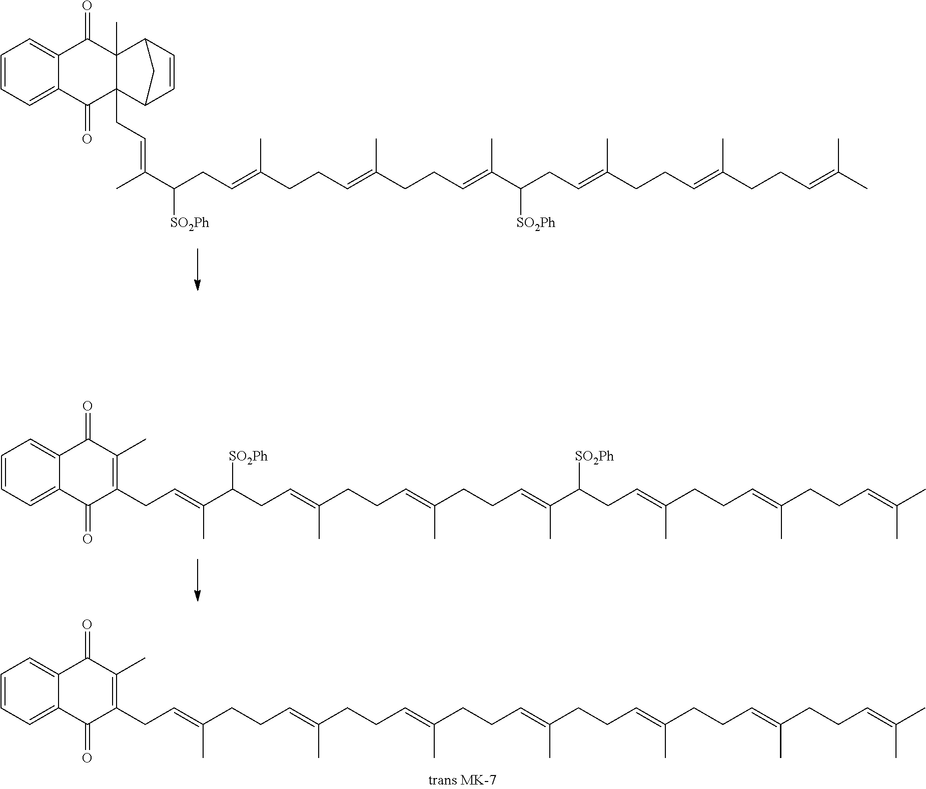

Furthermore, in any of the above schemes 1-4, the beginning of the process can be modified to use an —OR group instead of a —SO₂PH group in the starting isoprene or polyisoprene compound. The compound with the Br and OR functionalities can be coupled with the Diels Alder adduct via attack of the Br moiety. After coupling, the OR group can be converted to Br, and coupled with a further pentaprene or hexaprene —SO₂Ph compound. For scheme 4, for example, this would give:

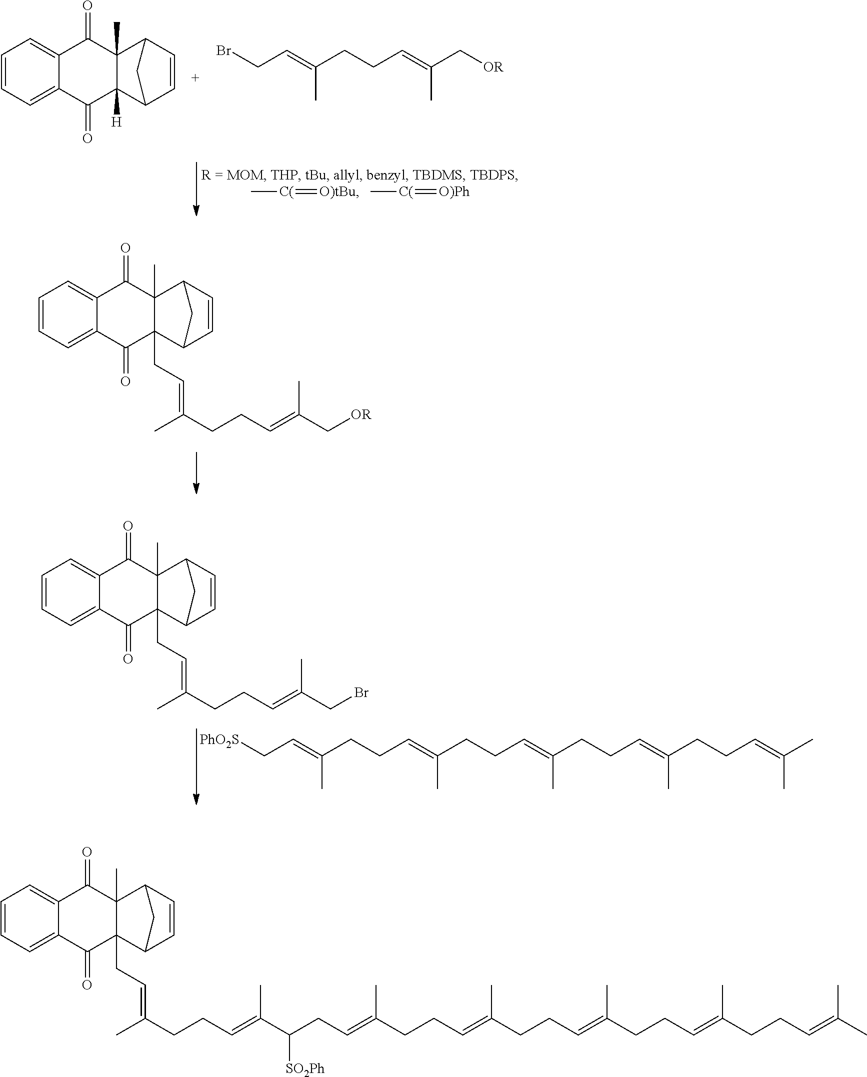

### Preparation of Polyprene Reagent

In a particular embodiment, X is Br in the polyprene reagent (III′):

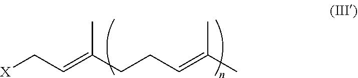

Compound (III′) with X═Br can be prepared upon reaction of the corresponding polyprenol and PBr₃. This can be made in situ and does not require isolation. In comparison, Teitelbaum et al. (Synthesis 2015, 47, 944-948) and Ji (Synthetic Communications 2003, 33:5, 763-772), use a purified bromide starting material.

The integer n is preferably 6, i.e. compound (III′) is preferably a heptaprene reagent.

X is a leaving group. Whilst Br is preferred, X may be selected from other halogens, mesylate, tosylate and triflate.

In a further embodiment, X is an ester, such as an ester of formula —COOR¹, with R¹ being a C1-C6 hydrocarbyl group, such as a linear or branched C1-C6 alkyl group or Ph. It is envisaged that such polyprene esters may react with the menadione Diels Alder adduct (II) in the presence of a base.

In a particular embodiment, compound (III′) is heptaprenylbromide, i.e.

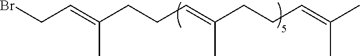

Heptaprenol is commercially available or can be synthesised according to WO2010/035000. Heptaprenyl bromide can be synthesised from heptaprenol using PBr₃, for example.

### Retro-Diels-Alder Reaction

A key benefit of the intermediate of formula (I) is that it can be converted to the final menaquinone upon simple heating. The final heating step also removes by-products as the cyclopentadiene by-product can be removed at low temperature.

Previous reactions have required the use of ammonium catalysts and acid (see Teitelbaum et al. (Synthesis 2015, 47, 944-948) and Ji (Synthetic Communications 2003, 33:5, 763-772)). Whilst the use of such reagents is possible, this is less favoured. These reagents are a cost and there is an increased risk of isomerization of bonds in the polyisoprene chains.

In a particular embodiment, upon heating or reacting with an ammonium catalyst and/or an acid, compound (I′)

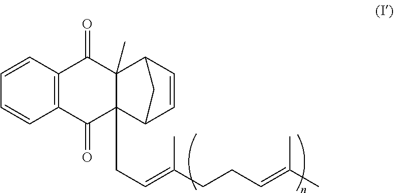

is converted by retro-Diels-Alder reaction to the desired menaquinone (VII).

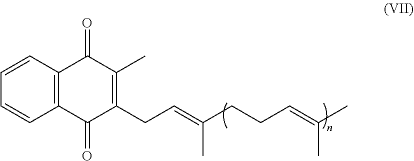

The retro-Diels-Alder reaction is accompanied by loss of cyclopentadiene.

The ammonium catalyst is typically a tetraalkylammonium salt, such as dodecyltrimethylammonium bromide. The acid is typically a carboxylic acid, such as acetic acid. Preferably, however, the retro-Diels-Alder reaction is carried out in the absence of an ammonium catalyst and acid.

In a particular embodiment, the retro-Diels Alder reaction is carried out in the absence of a dehydrogenating agent or oxidising agent.

In a particular embodiment, the retro-Diels Alder reaction, i.e. the conversion of a compound (I′) into a compound (VII), is carried out upon heating to a temperature in the range 50-150° C., such as 60-120° C., 70-110° C. or 80-100° C. It is surprising that this final retro-Diels Alder step can be performed upon simple heating, and is advantageous as it does not require additional reagents. The reaction is high yielding and achieves high purity. The only by-product, cyclopentadiene, with a boiling point of 40.8° C., is removed upon heating. Yields in the range of 80-95%, based on the starting polyprene reagent can be obtained.

The integer n is preferably 6, and thus in a preferred embodiment the invention provides a process comprising the following step:

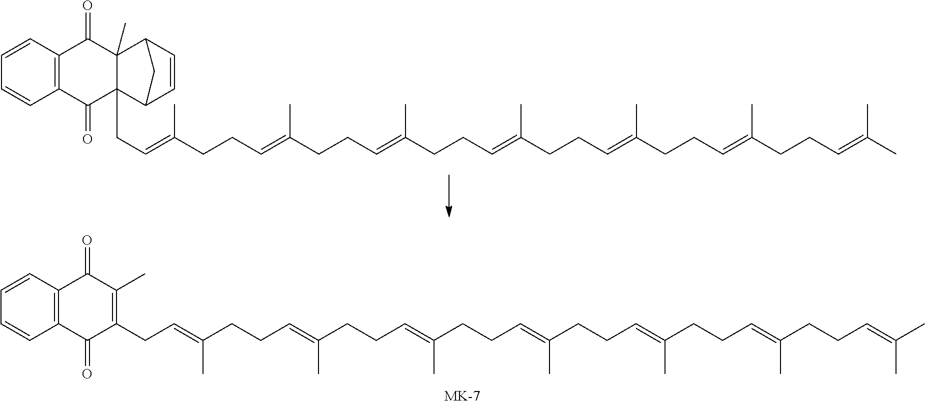

in particular wherein the retro Diels-Alder reaction above is effected by heating.

### One-Pot Method

In a particular embodiment, the invention provides a ‘one-pot’ method for preparing the target menaquinone of the present invention starting from a simple polyprene reagent. By ‘one-pot’ synthesis is meant a process whereby the starting reactants are subjected to successive chemical reactions in just one reactor without inter stage separation steps of the formed products. The reactor is preferably a flow reactor. The starting reactant in this case could be a polyprenol (e.g. heptaprenol) or a modified polyprene compound of formula (III) (e.g. heptaprenyl bromide). The one-pot method is highly advantageous as it avoids lengthy separation processes and purifications of the intermediate chemical compounds which can save time and resources while increasing chemical yield. Note therefore that in Teitelbaum each intermediate compound is isolated between steps.

In a particular embodiment, the invention provides a one pot process comprising the following steps:
i) reacting compound (III′)

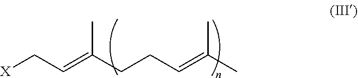

wherein X is a leaving group preferably selected from halogen (preferably Br), mesylate, tosylate and triflate;
and n is an integer between 2 and 10;
   with a compound (II):

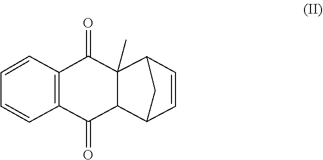 [SMILES: CC12C(=O)c3ccccc3C(=O)C1C1C=CC2C1]

in the presence of base (e.g. alkoxide such as a tert-butoxide, e.g. KO^{t}Bu).
ii) converting the resulting compound of formula (I′):

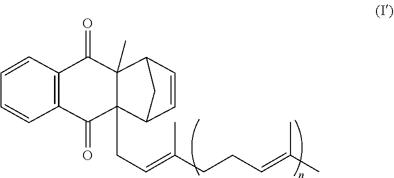

to the menaquinone product (VII)

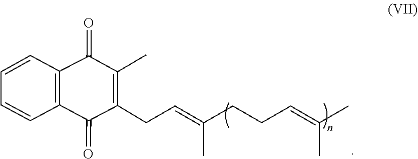

By a one pot process is meant that the product of step (i) is not isolated before step (ii) is carried out, e.g. is not isolated before the addition of the reagents required to effect step (ii). This last reaction can be performed by heating (preferred), or reaction with an ammonium catalyst and/or an acid.

In a further particular embodiment, the process of the invention comprises a one pot reaction in which in a first step i), wherein the polyprene alcohol of formula (VIII)

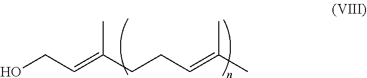

is converted to the polyprene reagent of formula

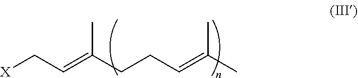

upon reaction with a suitable reagent. X is a leaving group selected from halogen, mesylate, tosylate and triflate. For X═Br, such a suitable reagent could be PBr₃.

In a second step ii), compound (III′) is then reacted with the menadione Diels adduct (II):

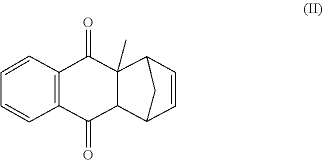 [SMILES: CC12C(=O)c3ccccc3C(=O)C1C1C=CC2C1]

in the presence of base (e.g. alkoxide such as a tert-butoxide, e.g. KO^{t}Bu).
In a third step iii), the resulting compound:

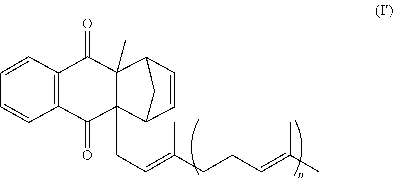

is then converted to the final menadione product.

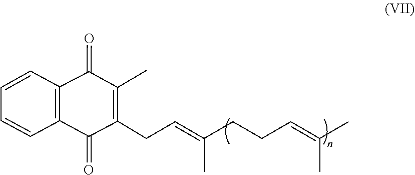

All these reactions are effected in a single pot without interstage isolation.

This latter reaction can be performed by heating (preferred), or reaction with an ammonium catalyst and/or an acid.

The advantage of this latter reaction sequence is that the entire reaction from the starting polyprenol can be carried out without the need for intermediate purifications.

In a further particular embodiment, the process is a flow process. Such a process can be effected in a flow reactor.

It will be appreciated that the reactions described herein may be carried out in an appropriate solvent. The skilled person can select solvents necessary. For example, the reaction of compounds of formula (II) and (III′) can be effected in a non-aqueous solvent such as THF. The retro Diels Alder can be effected in the absence of a solvent by just heating the material.

In a particularly preferred embodiment, the invention provides a process comprising steps (i) to (iii) or steps (ii) to (iii) in the schemes below.

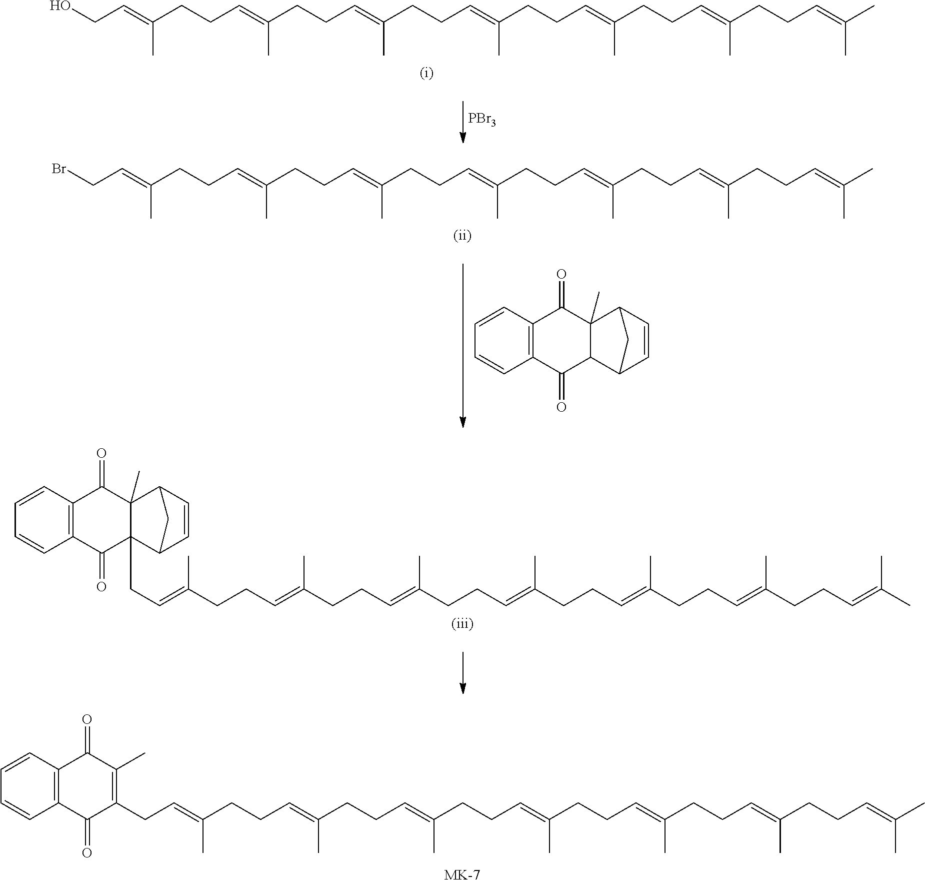

Step (iii) is preferably effected by heating. All steps are preferably carried out in a single pot, ideally a flow reactor.

The invention will now be explained with reference to the following non limiting examples.

### Example 1

### (1S,4R)-4a-((2E,6E,10E,14E,18E,22E)-3,7,11,15,19,23,27-heptamethyloctacosa-2,6,10,14,18,22,26-heptaen-1-yl)-9a-methyl-1,4,4a,9a-tetrahydro-1,4-methanoanthracene-9,10-dione (F)

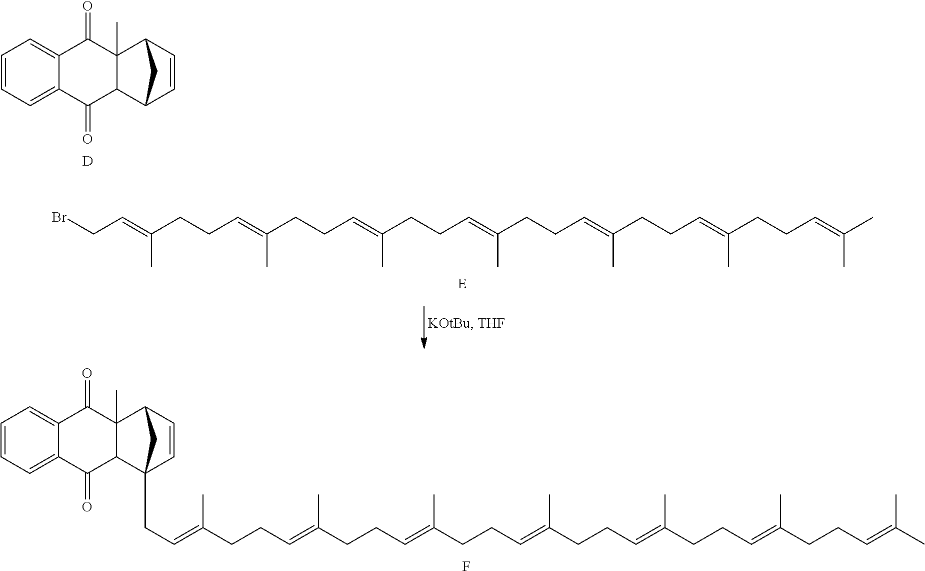

To a solution of heptaprenylbromide E (5.25 g, 9.42 mmol) in dry THF (25 ml) at 0° C. (ice bath), was added menadione adduct D (2.39 g, 10 mmol) and stirring continued for one minute before addition of potassium tert-butoxide (3.37 g, 30 mmol) in one portion. The cooling bath was removed and stirring continued for 30 minutes. The reaction mixture was quenched with 2 M HCl (25 ml), the aqueous layer extracted with heptane (2×25 ml). The combined organic extract was filtered through a short pad of silica gel (5 grams) and concentrated on the rotary evaporator to give the title compound (F) as a yellow oil.

### Example 2

### 2-((2E,6E,10E,14E,18E,22E)-3,7,11,15,19,23,27-heptamethyloctacosa-2,6,10,14,18,22,26-heptaen-1-yl)-3-methylnaphthalene-1,4-dione (G)

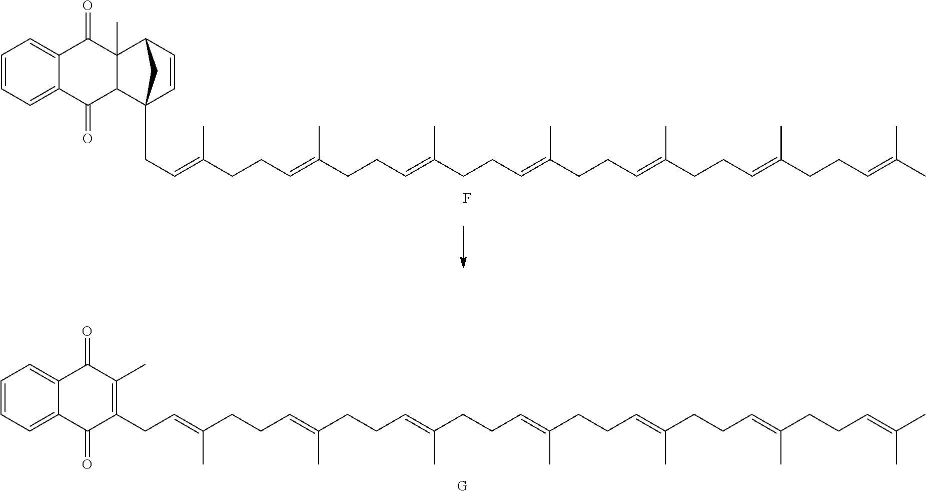

Compound F was heated at 90° C. for 30 min. After cooling to room temperature, the residue (6.33 g) was dissolved in acetone (50 ml) and cooled to −20° C. Left at this temperature overnight, filtered, washed with cold acetone and dried to give 5.06 g (83% from heptaprenol) of the title compound as a bright yellow solid.

### Example 3

### 2-((2E,6E,10E,14E,18E,22E)-3,7,11,15,19,23,27-heptamethyloctacosa-2,6,10,14,18,22,26-heptaen-1-yl)-3-methylnaphthalene-1,4-dione (G)

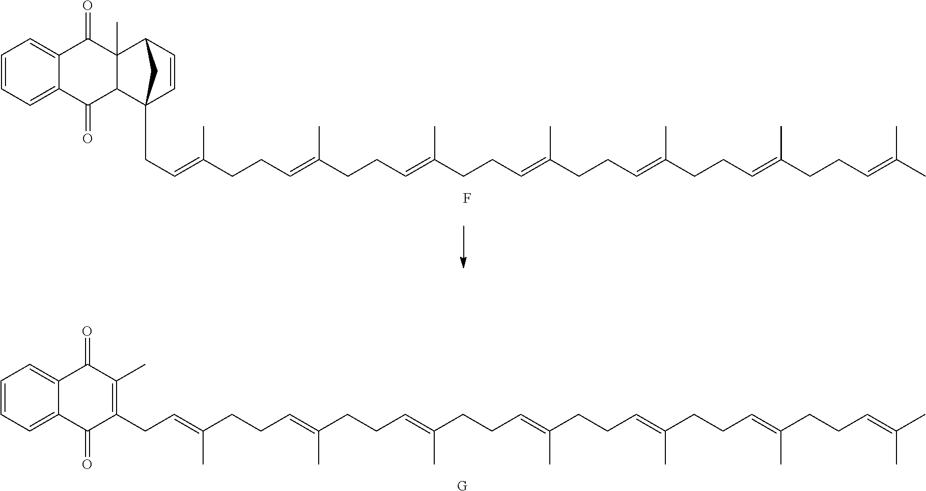

Compound F was dissolved in AcOH (0.1 mmol in 1.2 mL) followed by the addition of dodecyltrimethylammonium bromide (2 mg). The solution was heated to 90° C. for 60 min. After cooling to r.t., the solvent was evaporated, and the crude product was purified as above.

### Example 4

By similar reactions the following MK-6, MK-8 and MK-10 adducts and subsequently MK-6, MK-8 and MK-10 can be obtained:

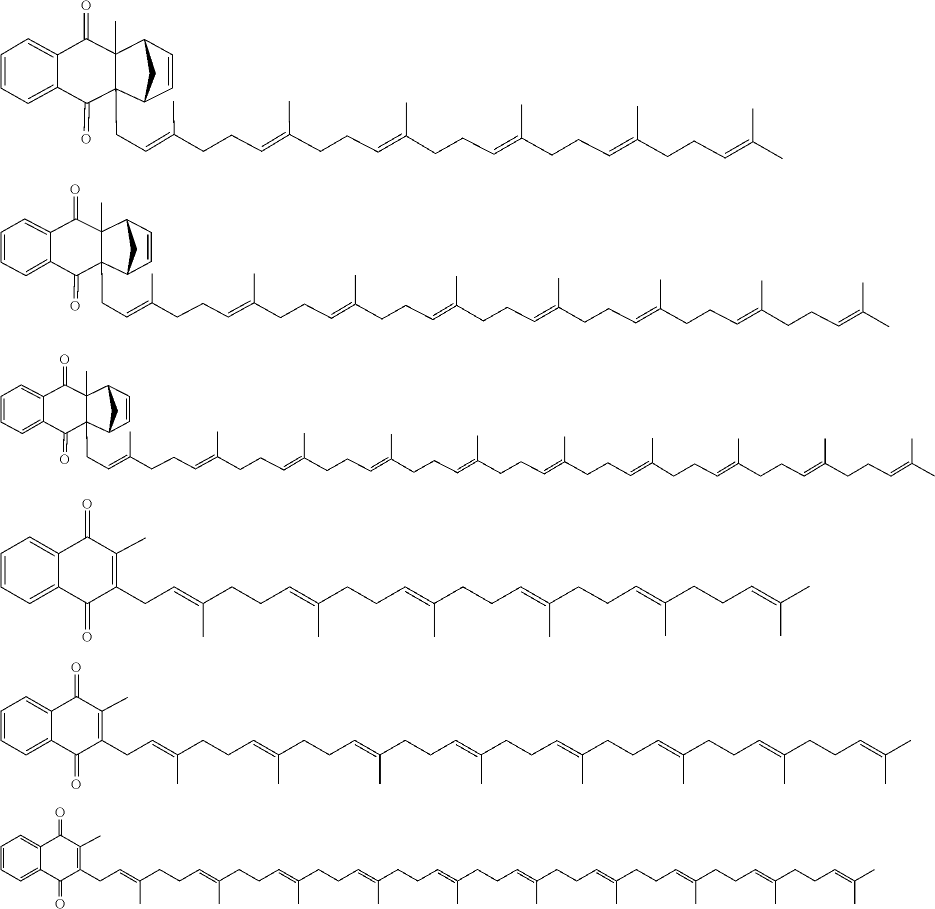

1. A process comprising converting a compound of formula (I′) 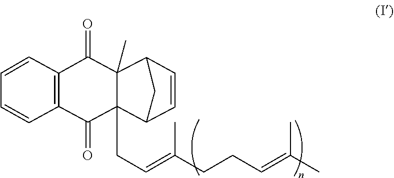
wherein n is an integer between 2 and 10 to a compound of formula (VII): 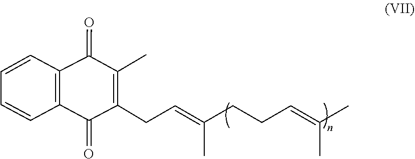
preferably by heating, especially in the absence of a solvent.

2. A process as claimed in claim 1, further comprising a step of forming the compound of formula (I′) by reaction of compound (II) with a compound of formula (III′) 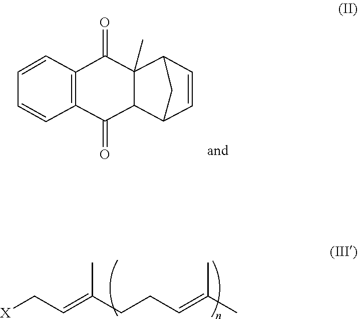
in the presence of a base;
wherein X is a leaving group preferably selected from halogen, mesylate, tosylate and triflate.

3. A process as claimed in claim 1, comprising carrying out the following steps in one pot:
i) reacting compound (III′) 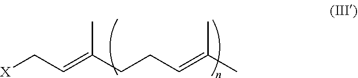
wherein X is a leaving group preferably selected from halogen (preferably Br), mesylate, tosylate and triflate;
and n is an integer between 2 and 10;
with a compound of formula (II): 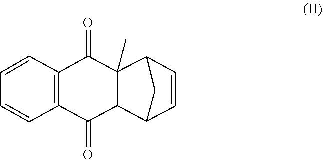 [SMILES: CC12C(=O)c3ccccc3C(=O)C1C1C=CC2C1]
in the presence of base (e.g. alkoxide such as a tert-butoxide, e.g. KO^{t}Bu);
ii) converting the resulting compound of formula (I′): 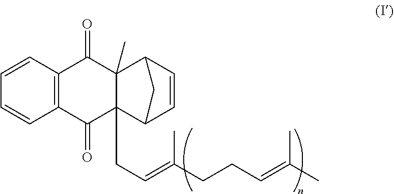
to the final menadione product (VIII) 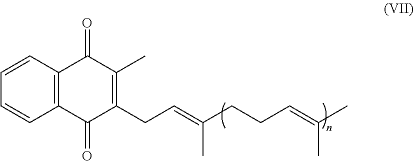
by heating, especially in the absence of a solvent.

4. A process as claimed in claim 1, comprising carrying out the following steps in one pot:
i) converting a polyprene alcohol of formula (VIII): 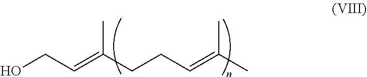
wherein n is an integer between 2 and 10;
into a polyprene reagent of formula (III′) 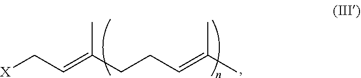
wherein X is Br, in the presence of PBr₃;
and
ii) reacting compound (III′) with compound (II): 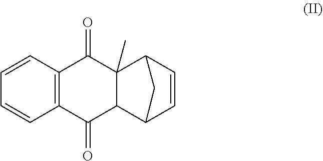 [SMILES: CC12C(=O)c3ccccc3C(=O)C1C1C=CC2C1]
in the presence of base (e.g. alkoxide such as a tert-butoxide, e.g. KO^{t}Bu).
iii) converting the resulting compound (I′): 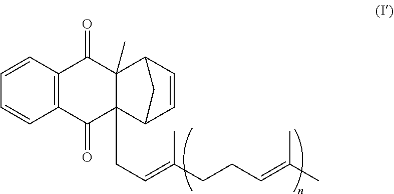
to the final menadione product 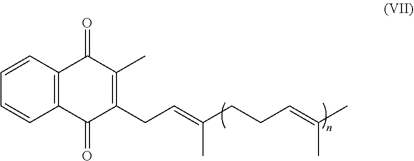
by heating, especially in the absence of a solvent.

5. A process as claimed in claim 3 or 4 wherein the one pot process occurs in a flow reactor.

6. A process as claimed in claim 1, wherein (I′) is obtained by
(i) reacting a compound of formula (IV): 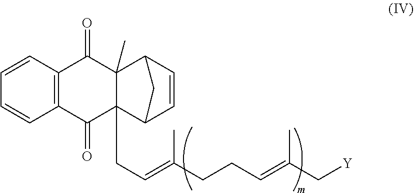
with a compound of formula (VI): 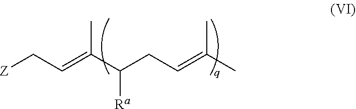
in the presence of a base,
wherein m+q=n−1;
n is 1 to 9;
m is an integer between 0 and 9;
q is an integer between 9 and 0;
Y is halide, hydroxyl or protected hydroxyl and Z is mesylate, tosylate, ester, —SPh or —SO₂Ph, or Y is mesylate, tosylate, ester, —SPh or —SO₂Ph and Z is halide, hydroxyl or protected hydroxyl;
R^{a} is independently H or a substituent selected from mesylate, tosylate, —SPh or —SO₂Ph;
and optionally in a step ii), the Z or Y group respectively and any non-H R^{a} group in the resulting compound are reduced to hydride(s).

7. The process 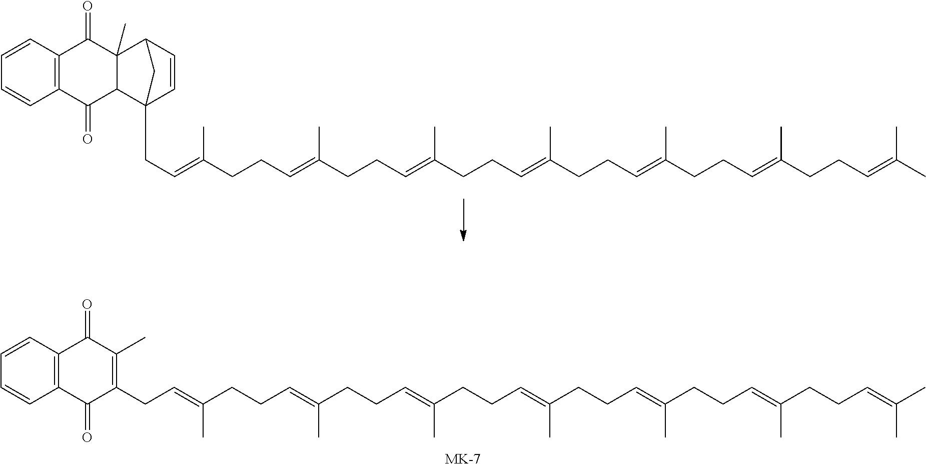
by heating, especially in the absence of a solvent.

8. A process comprising converting a compound of formula (I″) 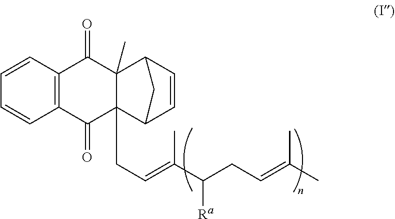
wherein n is an integer between 2 and 10
and wherein R^{a} is independently H or a substituent selected from mesylate, tosylate, —SPh or —SO₂Ph
to a compound of formula (VII″): 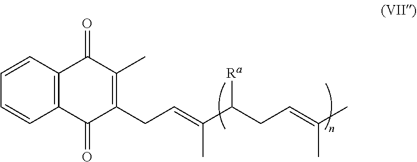
preferably by heating, especially in the absence of a solvent.

9. The process 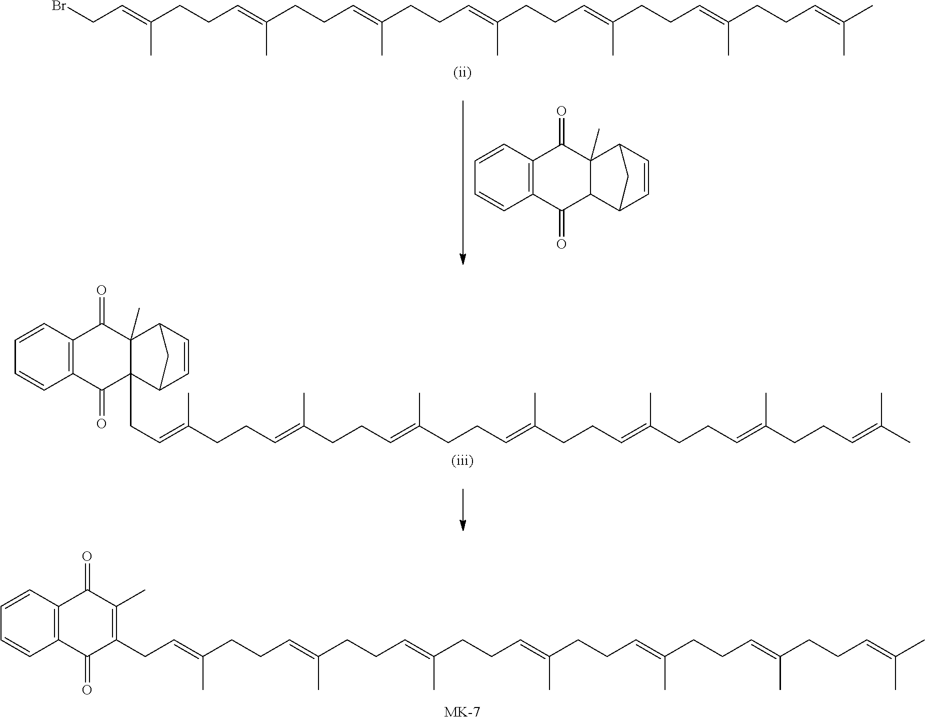
wherein step (iii) is effected by heating, especially in the absence of a solvent.

10. The process of claim 9, comprising the steps 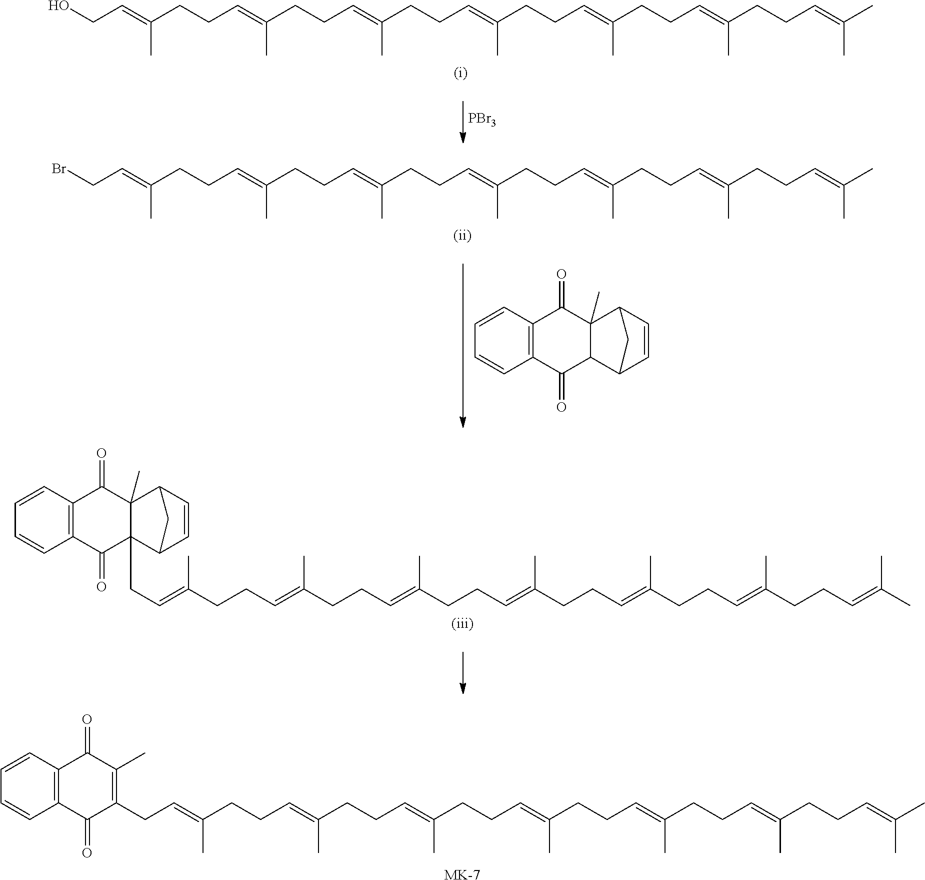
wherein step (iii) is effected by heating, especially in the absence of a solvent.

11. A compound of formula (I): 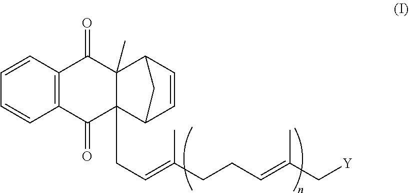
wherein n is an integer between 0 and 10;
Y is hydrogen, halide, mesylate, tosylate, —SPh, —SO₂Ph, ester, hydroxyl or protected hydroxyl;
with the proviso that if n=0 or 1, Y is not hydrogen.

12. A compound as claimed in claim 11, wherein Y is hydrogen and n is 2 to 10, preferably 5-10, 5-9, 5-8 or 5-7.

13. A compound as claimed in claim 11 or 12, wherein n is between 3 and 9, preferably 6.

14. A compound as claimed in any preceding claim of formula

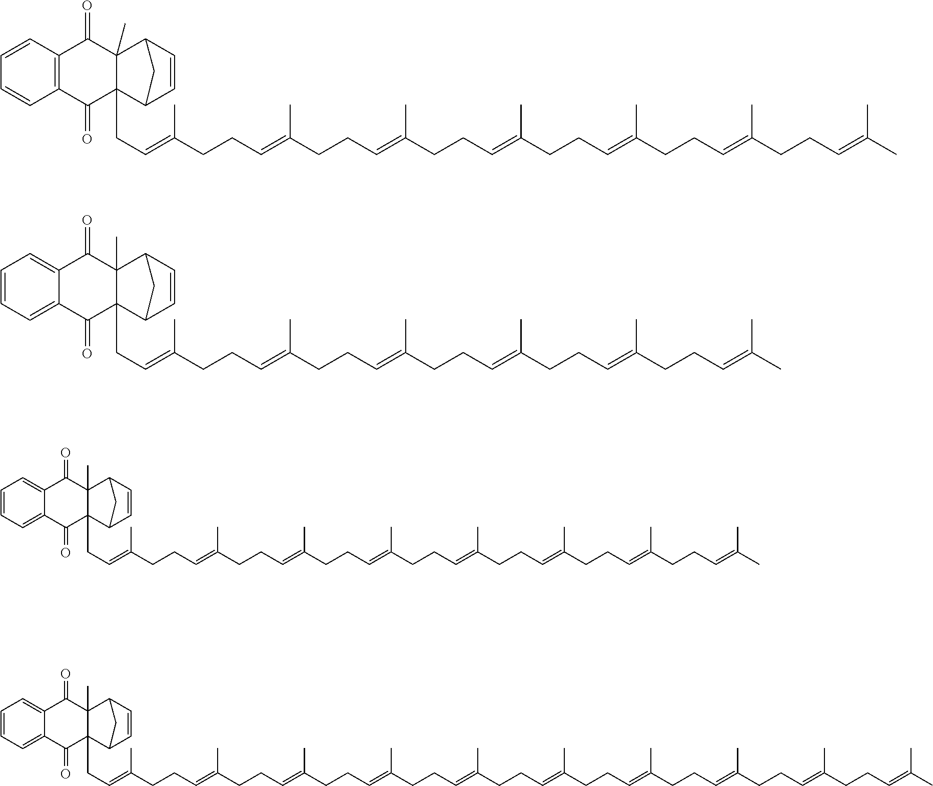 [SMILES: CC(C)=CCC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1 ; CC(C)=CCC/C(C)=C/CC/C(C)=C/CC/C(C)=C/CC/C(C)=C/CC/C(C)=C/CC12C(=O)c3ccccc3C(=O)C1(C)C1C=CC2C1 ; CC(C)=CCC/C(C)=C/CC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1 ; CC(C)=CCC/C(C)=C/CC/C(C)=C/CC/C(C)=C/CC/C(C)=C/CC/C(C)=C/CC/C(C)=C/CC/C(C)=C/CC/C(C)=C/CC/C(C)=C/CC12C(=O)c3ccccc3C(=O)C1(C)C1C=CC2C1]

15. A compound of formula (I″) 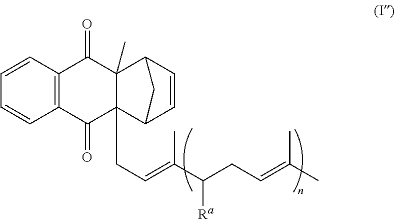
wherein n is an integer between 2 and 10
and wherein R^{a} is independently H or a substituent selected from mesylate, tosylate, —SPh or —SO₂Ph.

16. A compound of formula (I′″): 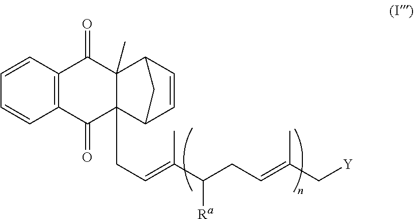
wherein R^{a} is independently H or a substituent selected from mesylate, tosylate, —SPh or —SO₂Ph;
Y is hydrogen, halide, mesylate, tosylate, —SPh, —SO₂Ph, ester, hydroxyl or protected hydroxyl
n is an integer between 0 and 10 when R^{a} is H, and n is an integer between 2 and 10 when Y is H;
with the proviso that if n=0 or 1, Y is not hydrogen.
